SEQUENCE LISTING (1 sequence)

SEQ ID NO: 1 - DNA, 700 bp, Pseudomonas sp. HN-72
ttgcgagcgtgctacagcagtcagcggatgacgcgagctcgctccctgattcagcggaggacgggtgagtaatgcctaggattctggctggtagtgggggacaacgtctcgataggaacgctaataccgcatacgtcctacgtgagatagcattagaccttcggaccttgcgctatcagatgagccttggtcggattagctagatggtgcagtaatggctcaggatggcgacgatccgtaactggtctgagaggatgatcactcacactggaactgagacacggtccaggctcctacgagagcgggcagtggtgaatattggacaatgggcgacagcctgatccaggcatgcagcgtgtgtgaagaaggtcttcggattgtaaagcactttaagttgtgaggaaggcgagtaagttaataccttgctgtcatgacgttaccgaaagaataagcaccggctaactctgagccagcagctgcggtaatacagatggtgcaagcgttaatcggaattactgggcgtatagcgcgcgtaggtggtttgttaagttggatgtgaaagccccgggctcaacctgggaactgaatccaccactggcaagctagagtacggtagagggtgctggaatatcctgtgtagcggtgaaatgcgtagatataggaaggaacaccagtggctacagcgaccacctggactgat

(19) United States
(12) Patent Application Publication
(10) Pub. No.: US 2010/0063318 A1
(43) Pub. Date: Mar. 11, 2010

(54) Purification Method of Crude Naphthalene Dicarboxylic Acid Using Microorganism and 2,6-Naphthalene Dicarboxylic Acid in Crystalline Form Obtained by Using the Same

(76) Inventors: Dong Sung Kim, Gwanyang-Dong (KR); Yong Bok Choi, Geumjeong-Dong (KR); Jong Hwan Lee, Cheoncheon-Dong (KR); Sung Kyoon Kim, Yaptap-Dong (KR); Ik Hyun Kwon, Dangsan-Dong (KR)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/094,513

(22) PCT Filed: Feb. 14, 2006
(86) PCT No.: PCT/KR2006/000519

§ 371 Date: Oct. 27, 2008

(30) Foreign Application Priority Data
Dec. 12, 2005 (KR) 10-2005-0121682

(51) Int. Cl. C07C 63/38; C12P 7/44

(52) U.S. Cl. 562/488; 435/136

(57) ABSTRACT

Disclosed is a method for purifying a crude naphthalene dicarboxylic acid using microorganism. According to the purification method, a crude naphthalene dicarboxylic acid is purified by reacting a microorganism having the ability to convert 2-formyl-6-naphthoic acid to 2,6-naphthalene dicarboxylic acid with a crude naphthalene dicarboxylic acid, adding an acidic solution to the reaction solution under particular conditions, stirring the mixed solution to crystallize the crude naphthalene dicarboxylic acid, washing the crystallized crude naphthalene dicarboxylic acid, and drying the washed product to obtain 2,6-naphthalene dicarboxylic acid in a pure crystalline form. Advantageously, the purification method enable production of high-purity crystalline 2,6-naphthalene dicarboxylic acid on an industrial scale in an environmentally friendly manner.

### TECHNICAL FIELD

The present invention relates to a method for purifying a crude naphthalene dicarboxylic acid using a microorganism and 2,6-naphthalene dicarboxylic acid in a crystalline form produced by the method. More particularly, the present invention relates to a method for purifying a crude naphthalene dicarboxylic acid by reacting a microorganism having the ability to convert 2-formyl-6-naphthoic acid to 2,6-naphthalene dicarboxylic acid with a crude naphthalene dicarboxylic acid to remove 2-formyl-6-naphthoic acid contained as an impurity in the crude naphthalene dicarboxylic acid, adding an acidic solution to the reaction solution under particular conditions, stirring the mixed solution to crystallize the crude naphthalene dicarboxylic acid, washing the crystallized crude naphthalene dicarboxylic acid to remove other impurities contained in the crystallized crude naphthalene dicarboxylic acid, and drying the washed product to obtain 2,6-naphthalene dicarboxylic acid in a pure crystalline form.

### BACKGROUND ART

2,6-Naphthalene dicarboxylic acid (NDA) and its diester (i.e. 2,6-naphthalene dicarboxylate (NDC)) are useful monomers for the preparation of a variety of polymeric materials, such as polyester and polyamide. For example, NDA and NDC can be condensed with ethylene glycol to form poly(ethylene 2,6-naphthalate) (PEN), which is a high-performance polyester material. Fibers and films made from PEN exhibit high strength and superior thermal properties, compared to those made from poly(ethylene terephthalate) (PET). Based on these advantages, PEN is highly suitable for use in the production of commercial articles, such as thin films, which can be used for the manufacture of magnetic recording tapes and electronic components. In addition, since PEN is highly resistant to the diffusion of gases, particularly carbon dioxide, oxygen and water vapor, films made from PEN are useful for the manufacture of food containers, especially hot-fill food containers. PEN can also be used to produce reinforced fibers useful for the manufacture of tire cords.

NDC is currently produced by oxidizing 2,6-dimethylnaphthalene (2,6-DMN) to obtain a crude naphthalene dicarboxylic acid (cNDA) and esterifying the cNDA. At present, NDC is used as a major raw material for the synthesis of PEN. However, some problems are presented when NDC is used as a raw material for the synthesis of PEN, compared to when 2,6-naphthalene dicarboxylic acid (NDA) is used. Firstly, water is formed as a by-product during the condensation of NDA, whereas methanol is formed as a by-product in the case of NDC, thus risking the danger of explosion. Secondly, since pure NDC is produced by esterifying NDA and purifying the esterification product, one additional processing step is involved, compared to the use of NDA. Thirdly, the use of NDC is not suitable in view of the use of existing PET production facilities. Despite the problems associated with the use of NDC, NDC is preferentially used to produce PEN because it is still difficult to produce purified NDA having a purity necessary for the synthesis of PEN.

2,6-Dimethylnaphthalene (2,6-DMN) is oxidized to form a cNDA containing various impurities, such as 2-formyl-6-naphthoic acid (FNA), 2-naphthoic acid (NA) and trimellitic acid. Particularly, the presence of FNA in a cNDA stops the polymerization for the production of PEN, resulting in a low degree of polymerization. This low degree of polymerization adversely affects the physical properties of the final polymer (i.e. PEN) and causes coloration of the polyester. It is thus essential to remove FNA present in a cNDA, but difficulties exist in removing FNA.

Under these circumstances, research has been conducted on various chemical methods for the removal of FNA present in a cNDA or purification of NDA. For example, NDA is produced by i) recrystallizing a cNDA, ii) oxidizing a cNDA one more time, or iii) treating a cNDA with methanol to produce NDC and hydrating the NDC. Further, purified NDA is produced by hydrogenation of a cNDA. In addition to these chemical methods, many processes, e.g., solvent treatment, melting/crystallization, high-pressure crystallization and supercritical extraction, have been employed to purify NDA.

For example, U.S. Pat. No. 5,859,294 discloses a process for the production of a naphthalene dicarboxylic acid, which comprises dissolving a crude naphthalene dicarboxylic acid in an aqueous solution containing an aliphatic or alicyclic amine, removing heavy metal components contained as impurities until the content of the heavy metal components based on the crude naphthalene dicarboxylic acid is 100 ppm or less, and heating the aqueous solution containing a naphthalene dicarboxylic acid amine salt to provide a high-purity naphthalene dicarboxylic acid by distilling off the amine.

U.S. Pat. No. 6,255,525 discloses a process for preparing an aromatic carboxylic acid having improved purity comprising the steps of contacting a mixture comprising an impure aromatic carboxylic acid and water at a pressure of 77 to 121 kg/cm² and a temperature of 277 to 316° C. in the presence of hydrogen with a carbon catalyst which is free of a hydrogenation metal component, cooling the mixture to form crystallized aromatic carboxylic acid, and recovering the crystallized aromatic carboxylic acid from the cooled mixture.

In connection with the crystallization reaction of NDA, U.S. Pat. No. 6,087,531 teaches a process for recovering a naphthalene dicarboxylic acid (NDA) crystal comprising the steps of dissolving poly(alkylene naphthalene dicarboxylate) in an aqueous basic solution (e.g., an aqueous solution of an alkali metal base, an aqueous hydroxide solution or an aqueous solution of an alkali metal carbonate) at a temperature of 125 to 400° C., neutralizing the aqueous solution with an acid at 170 to 240° C., and recovering the NDA. For example, an NDA crystal is recovered by dissolving the polyester material in a NaOH or KOH solution at a temperature of 125 to 400° C., neutralizing the aqueous solution with acetic acid, and recovering the NDA.

U.S. Pat. No. 6,426,431 teaches a method of increasing the purification yield of 2,6-NDA by as high as 45%, the method comprising treating an aqueous solution of K₂-NDA at a CO₂ pressure of 0-200 psi and a temperature of 0-50° C. to form KH-NDA, suspending the KH-NDA in water in a weight ratio higher than 1:8 (KH-NDA:water), and further treating the suspension at a temperature above 100° C. (140-160° C.) and at a CO₂ pressure above 100 psi (175-250 psi).

However, since these processes require high-temperature and high-pressure conditions, time-consuming treatments and use of large amounts of expensive materials to produce a high-purity NDA crystal, they are economically disadvantageous. Other disadvantages of the processes are very low purification yield and purity of NDA and occurrence of aggregation between individual NDA crystal particles, which makes the processes difficult to practice industrially.

### DISCLOSURE OF INVENTION

### Technical Problem

Therefore, the present invention has been made in view of the problems of the prior art, and it is one object of the present invention to provide a method for efficiently producing high-purity NDA in high yield by purifying and crystallizing a crude naphthalene dicarboxylic acid at ambient pressure and temperature conditions using a microorganism capable of converting FNA to NDA.

It is another object of the present invention to provide a high-purity NDA crystal with a uniform size produced by the method.

### Technical Solution

In accordance with one aspect of the present invention for achieving the above objects, there is provided a method for purifying a crude naphthalene dicarboxylic acid using a microorganism, the method comprising the steps of (a) reacting a microorganism having the ability to convert 2-formyl-6-naphthoic acid to 2,6-naphthalene dicarboxylic acid with a crude naphthalene dicarboxylic acid to remove 2-formyl-6-naphthoic acid contained in the crude naphthalene dicarboxylic acid, (b) adding an acidic solution to the reaction solution prepared in step (a) to adjust the pH of the reaction solution and reacting the mixed solution with stirring to crystallize the crude naphthalene dicarboxylic acid, (c) washing the crystallized crude naphthalene dicarboxylic acid to remove impurities contained in the crystallized crude naphthalene dicarboxylic acid, and (d) drying the washed product to obtain 2,6-naphthalene dicarboxylic acid in a pure crystalline form.

In accordance with another aspect of the present invention, there is provided 2,6-naphthalene dicarboxylic acid in a pure crystalline form produced by the purification method.

### Advantageous Effects

According to the purification method of the present invention, since a crude naphthalene dicarboxylic acid is purified and crystallized under respective suitable conditions using a microorganism capable of converting FNA to NDA, high-purity crystalline 2,6-naphthalene dicarboxylic acid can be advantageously produced on an industrial scale in an environmentally friendly manner.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is shows a partial sequence of 16S rDNA of Pseudomonas sp. strain HN-72 used in a method of the present invention;

FIG. 2 is a micrograph of a crystallized cNDA obtained in a third step of Example 1 according to the present invention;

FIGS. 3a, 3b and 3c are micrographs of cNDA crystals obtained by adding a sulfuric acid solution to reaction solutions of a purified cNDA and stirring the mixed solutions at different rates of 50 rpm, 200 rpm and 800 rpm, respectively, in Experimental Example 1 of the present invention;

FIGS. 4a, 4b and 4c are micrographs of cNDA crystals obtained by adding a sulfuric acid solution to reaction solutions of a purified cNDA and stirring the mixed solutions at different reaction temperatures of 15° C., 40° C. and 80° C., respectively, in Experimental Example 2 of the present invention; and

FIG. 5 shows a purification system for implementing a method for purifying a crude naphthalene dicarboxylic acid according to an embodiment of the present invention.

### BEST MODE FOR CARRYING OUT THE INVENTION

A more detailed explanation of the respective steps of the method according to the present invention will be provided below.

(i) First Step: Purification Using Microorganism

In this step, a microorganism having the ability to convert 2-formyl-6-naphthoic acid to 2,6-naphthalene dicarboxylic acid is reacted with a crude naphthalene dicarboxylic acid to convert 2-formyl-6-naphthoic acid contained in the crude naphthalene dicarboxylic acid to 2,6-naphthalene dicarboxylic acid.

The first step includes the sub-steps of 1) inoculating the microorganism onto a liquid medium, culturing the inoculum with shaking, collecting the cultured bacteria by centrifugation, and suspending the collected bacteria in physiological saline or distilled water to activate the bacteria, 2) mixing a crude naphthalene dicarboxylic acid (cNDA) as a matrix with a buffer solution and adjusting the pH of the mixed solution by the addition of an alkaline solution to prepare a reaction solution for subsequent purification, and 3) reacting the active bacteria prepared in 1) with the reaction solution prepared in 2) to convert 2-formyl-6-naphthoic acid contained in the crude naphthalene dicarboxylic acid to 2,6-naphthalene dicarboxylic acid, so that the purity of the 2,6-naphthalene dicarboxylic acid is increased.

Any microorganism may be used without any particular limitation if it has the ability to decompose 2-formyl-6-naphthoic acid. A microorganism belonging to the genus Bacillus or Pseudomonas is preferably used in the method of the present invention.

Most preferred is Bacillus sp. F-1 (KCTC-10342BP), Bacillus sp. F-3 (KCTC-10335BP), or Pseudomonas sp. HN-72 (KCTC-10819BP). Bacillus sp. F-1 and Bacillus sp. F-3 are described in Korean Patent Application No. 2002-0087819, and Pseudomonas sp. strain HN-72 was deposited at GenBank of the Korea Research Institute of Bioscience and Biotechnology (KRIBB), which is an international depository authority, under the accession number of KCTC-10819BP on Jun. 21, 2005.

These microorganisms can be easily cultured in a liquid medium (e.g., an LB or M9 medium) in a wide temperature range of 25 to 45° C. and preferably 28 to 35° C.

The kind of the buffer solution is not especially restricted. As the buffer solution, there can be used, for example, water, sodium carbonate buffer (Na₂O₃/NaHCO₃), glycine buffer (glycine/NaOH), potassium phosphate buffer (KH₂PO₄/KOH), sodium phosphate buffer (Na₂HPO₄/NaH₂PO₄), succinic acid buffer (succinic acid/NaOH), sodium acetate buffer (sodium acetate/acetic acid), citric acid buffer (citric acid/sodium citrate), sodium pyrophosphate buffer (Na₄PO₄/HCl), boric acid buffer (boric acid/NaOH), or sodium borate buffer (sodium borate/HCl). Preferred are potassium phosphate buffer (KH₂PO₄—KOH) and boric acid buffer (boric acid/NaOH), which have a pH range of 6.0 to 9.0. It is preferred that the buffer solution have a concentration of 0.01 to 100 mM. The alkaline solution is preferably a NaOH or KOH solution, but is not limited thereto. The pH of the mixed solution is preferably adjusted to the range of 6 to 10.

In the second sub-step, an organic solvent may be additionally added to the mixed solution for the purpose of dissolving the cNDA. Examples of preferred organic solvents include dimethylsulfoxide (DMSO), N,N-dimethylformamide (DMF), N,N-dimethylacetamide (DMA), and tetrahydrofuran (THF). Of these, dimethylsulfoxide is most preferred in terms of enzymatic activity. The organic solvent is preferably added at a concentration of 0.01 to 10%. More preferably, no organic solvent is added. The addition of the organic solvent at a concentration exceeding 10% causes lysis of the cell membranes of the microorganism, resulting in inhibition of the reaction.

In the third sub-step, the reaction is preferably conducted at 25 to 50° C. for 1 minute to 2 hours and more preferably at 35 to 40° C. for 25 to 40 minutes. When the reaction temperature is lower than 25° C. or higher than 50° C., a marked decrease in the activity of the bacteria is undesirably caused.

On the other hand, there is an intimate relationship among the FNA content in the cNDA, the concentration of the cNDA in the reaction solution and the amount of the bacteria necessary to completely remove the FNA. That is, as the FNA content in the cNDA and the concentration of the cNDA in the reaction solution increase, a larger amount of the bacteria is required to remove the FNA.

(ii) Second Step: Crystallization

In this step, an acidic solution is added to the reaction solution prepared in the first step to adjust the pH of the reaction solution, and is then reacted with the reaction solution with stirring to crystallize the crude naphthalene dicarboxylic acid.

More specifically, an appropriate amount of an acidic solution is added to the reaction solution prepared in the first step in a reactor equipped with an agitator to adjust the pH of the reaction solution to a desired range, and then the mixed solution is reacted with continuous stirring while maintaining the temperature of the mixed solution at a constant level, so that the cNDA in an amorphous form present in the FNA-free reaction solution is crystallized at ambient pressure and temperature conditions.

Since this crystallization enables production of a cNDA crystal having a uniform size of 100 □ or more at ambient pressure and temperature conditions, the purification method of the present invention is economically advantageous in terms of production cost and processing. In addition, since no aggregation between the individual cNDA crystal particles occurs, the purification method of the present invention is suitable for actual use and is advantageous in terms of a high recovery rate of the final product.

As the acidic solution, there may be used, for example, sulfuric acid, hydrochloric acid, glacial acetic acid or nitric acid. The use of sulfuric acid or nitric acid is preferred because it leads to large size and high yield of the cNDA crystal. That is, the addition of sulfuric acid or nitric acid enables the production of a cNDA crystal having a uniform size of 100 □ or more in high yield.

The pH of the reaction solution is preferably adjusted to 1-4 to increase the recovery rate of the final product. As the pH of the reaction solution decreases, the recovery rate of the final product tends to increase from about 92% to about 99.9%.

The crystallization is performed at about 4° C. to about 80° C. and preferably 25° C. to 60° C. The crystallization is performed for about 1 minute to about 12 hours and more preferably 2 to 30 minutes in view of continuous processing. Too short a time of the crystallization may cause a low degree of crystallization of the cNDA, leading to aggregation between the individual cNDA crystal particles upon polymerization. Meanwhile, too long a time of the crystallization results in an unnecessary energy loss.

The stirring required to perform the crystallization of the cNDA is typically performed at a rate of 0 to 1,000 rpm and preferably 0 to 400 rpm.

(iii) Third Step: Washing

In this step, the crystallized crude naphthalene dicarboxylic acid obtained in the second step is washed to remove impurities contained therein.

Although the FNA is converted to NDA and is completely removed through the purification reaction using the microorganism and the crystallization reaction, other impurities, including 2-naphthoic acid (NA), methylnaphthoic acid (MNA) and trimellitic acid (TLMA), remain unremoved. When the crystallized cNDA is dispersed in water and washed several times under given conditions to completely remove the remaining impurities. The complete removal of the impurities is based on a difference in solubility of the cNDA and the impurities in water. At this time, the microorganism used to purify the cNDA is also removed by the washing.

In this step, it is desirable to practice the separation in a state in which the reaction by-products are dissolved in the solvent and NDA is precipitated as much as possible. The temperature range for the separation is between 100 and 250° C. and preferably between 150 and 230° C. The separation of the solvent at a temperature higher than 250° C. causes increased loss of the purified NDA, leading to a considerable drop in yield. Meanwhile, the separation of the solvent at a temperature lower than 100° C. unfavorably causes low dissolution of the impurities, including NA, MNA and TLMA.

More specifically, the crystallized cNDA is dispersed in water, stirred at a pressure of 1 to 28 kg/cm² and a temperature of 100 to 250° C. for 10 minutes to 1 hour, and filtered to remove the water. This procedure may be repeated several times to remove the impurities. At this time, the solvent is preferably used in an amount of 5 to 20 times of the weight of the crystallized cNDA.

(iv) Fourth Step: Drying

In this step, the NDA crystal, from which the impurities are removed from the crystallized cNDA by the washing, is dried at a specified temperature to obtain NDA in a pure crystalline form. At this time, the drying is preferably performed at 30 to 200° C. The drying is performed, without limitation, by a conventional technique known in the art.

The method of the present invention may further comprise the step of removing the microorganism used in the first step after the first step and prior to the second step.

The previous removal of the microorganism after the purification of the cNDA and prior to the crystallization of the cNDA can contribute to improvements in purity and recovery rate of the NDA crystal.

The removal of the microorganism is achieved, without limitation, by a conventional technique known in the art. For example, a microfilter system, a continuous type centrifugal separator or a decanter may be used to remove the microorganism. The microfilter system uses a filter having a pore size of 0.1 to 0.5 □ and made of a material selected from ceramics, stainless steel, polypropylene and polyethylene terephthalate (PET).

In another aspect, the present invention provides 2,6-naphthalene dicarboxylic acid in a pure crystalline form produced by the purification method.

The purification method of the present invention enables the production of high-purity 2,6-naphthalene dicarboxylic acid in a high yield of 99.8% or higher. Depending on the intended applications and needs, the treatment conditions may be varied to produce 2,6-naphthalene dicarboxylic acid in a regular or random crystalline form. The 2,6-naphthalene dicarboxylic acid in a regular crystalline form may have a lattice structure.

The 2,6-naphthalene dicarboxylic acid crystal of the present invention has an average particle diameter not smaller than 100 □ and a uniform particle shape. Accordingly, the 2,6-naphthalene dicarboxylic acid crystal of the present invention is very suitable for the formation of a low-viscosity slurry with ethylene glycol when it is polymerized with the ethylene glycol to produce PEN. A 2,6-naphthalene dicarboxylic acid crystal having an average particle diameter smaller than 100 □ is difficult to treat and exhibits a poor ability to form a slurry with ethylene glycol when it is mixed with the ethylene glycol to prepare PEN, causing increased power consumption. The 2,6-naphthalene dicarboxylic acid crystal of the present invention preferably has an average particle diameter of 100 to 150 □.

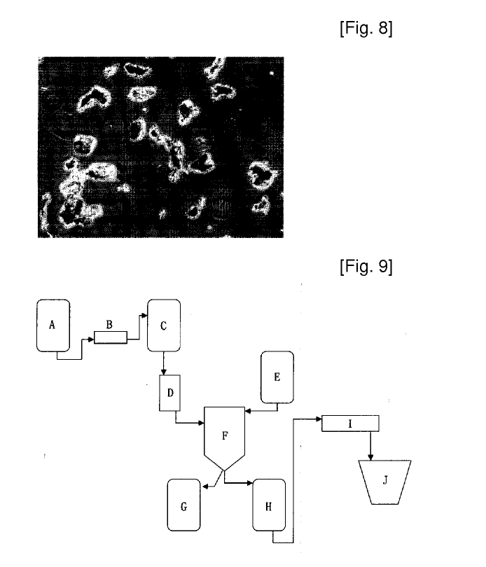

FIG. 5 shows a purification system for implementing a method for purifying a crude naphthalene dicarboxylic acid according to an embodiment of the present invention. With reference to FIG. 5, the purification method according to the embodiment of the present invention will be explained in more detail below.

First, a specified amount of a buffer solution is introduced into a reactor A where a crude naphthalene dicarboxylic acid is purified by a microorganism. A cNDA is added to the reactor, and subsequently, an alkaline solution is added to the reactor with stirring to adjust the pH of the reaction solution. A specified amount of water is added to the reactor to prepare a reaction solution for subsequent purification. Active bacteria are added to the reaction solution to react them with the reaction solution while maintaining the temperature of the reaction solution at a constant level. By this procedure, FNA contained in the cNDA is converted to NDA in the reactor A, and can be finally removed.

After completion of the reaction, the reaction solution is passed through a unit B where the microorganism used to remove the FNA is removed. The removal of the microorganism using the unit B may be omitted, if needed, because the removal of the microorganism can be achieved in a downstream filtering/cleaning unit F.

The reaction solution, from which the microorganism is removed, is transferred to a crystallization reactor C. An acidic solution is added to the crystallization reactor C with stirring to conduct a crystallization reaction.

A slurry containing the crystallized cNDA is obtained after the crystallization reaction. The slurry is heated to about 100 to about 250° C. using a preheater D, and is then fed into a filtering/cleaning unit F where a heater is provided to maintain the temperature of the slurry at 100 to 250° C. The pressure of the filtering/cleaning unit F is maintained at 1 to 25 kg/cm². The filtering/cleaning unit F is equipped with a filter (pore size: 10-100 □).

The filtering/cleaning unit F is connected to a high-pressure filtrate collection unit

G. A filtrate is discharged from the filtering/cleaning unit F into the high-pressure filtrate collection unit G and solid components are filtered by the filter included in the filtering/cleaning unit F.

Water (100-250° C.) preheated in a solvent heating/supply unit E is supplied to the filtering/cleaning unit F. After stirring is continued in the filtering/cleaning unit F for a given time, a secondary filtrate is discharged into the high-pressure filtrate collection unit G. If necessary, the cleaning step is repeated once or twice. Pure NDA remaining after the cleaning is mixed with preheated water (100-250° C.) to form a slurry. The slurry is sent to a high-pressure slurry collection unit H via a slurry discharge line. After the pressure of the high-pressure slurry collection unit H drops to ambient pressure, the slurry containing the pure NDA is transferred to a powder separation unit I where the solvent is removed from the slurry, followed by drying in a drying unit J to collect the pure NDA only.

### Mode for the Invention

Hereinafter, the constitution and effects of the present invention will be specifically explained with reference to the following examples. However, these examples are given for the purpose of illustration and are not to be construed as limiting the scope of the invention.

### Example

### Example 1

First Step: Screening of Microorganism

(1) Isolation of Strain

Soil was sampled from wastewater treatment plants, oil reservoirs and gas stations located in Gyeonggi Do, Korea. 5 g of each of the soil samples was added to 50 ml of a 0.85% physiological saline solution, shaken and filtered to obtain a filtrate. The filtrate was diluted to an appropriate level, plated on an LB solid medium containing cNDA, and cultured in an incubator at 30° C. As a result of the culture, 200 microorganisms were isolated.

Only microorganisms that decompose 2-naphthaldehyde having a formyl group at the same position as that of FNA were primarily screened in accordance with the following procedure. First, each of the 200 microorganisms was inoculated onto 1 ml of an LB liquid medium and cultured with shaking at 200 rpm and 30° C. for 16 hours. The cultures were centrifuged to collect the corresponding bacteria. The collected bacteria were suspended in 1 ml of a 0.85% physiological saline solution. Separately, 0.2 ml of a 2-naphthaldehyde solution (50 □/ml in DMSO) was added to sample cuvettes containing 3 ml of a β-NAD solution (0.25 mg/ml in 100 mM KH₂PO₄—KOH (pH 8.0)), and 0.2 ml of DMSO was added to a blank cuvette containing 3 ml of a β-NAD solution (0.25 mg/ml in 100 mM KH₂PO₄—KOH (pH 8.0)). The cuvettes were placed in respective spectrometer and stabilized for 3 minutes. 5 minutes after the addition of 0.05 ml of the microorganism suspensions to the respective sample cuvettes, the absorbance of the mixtures was measured at 340 nm. The absorbance values of the mixtures in the sample cuvettes were compared with the absorbance value of the blank cuvette to confirm the oxidation of the formyl group of the 2-naphthaldehyde to a carboxyl group. As a result, four microorganisms having an absorbance difference of a minimum of 0.1 were isolated.

Each of the four primarily screened microorganisms was inoculated onto an LB liquid medium and cultured with shaking at 200 rpm and 30° C. for 16 hours. The cultures were centrifuged to collect the corresponding bacteria, washed with a 0.85% physiological saline solution, and reacted with solutions having the composition indicated in Table 1 in a reaction bath at 30° C. for 3 hours. The reaction products were analyzed by high-performance liquid chromatography (HPLC) to finally screen a strain that was highly capable of removing FNA. The HPLC analysis was performed under the conditions indicated in Table 2. The HPLC analysis shows that the strain termed HN-72 was highly capable of decomposing FNA.

TABLE 1
Reaction solution for final screening

| | Volume | |
|---|---|---|
| Composition | (ml) | Remarks |
| 50 mM KH₂PO₄—KOH (pH 8.0) | 37.5 | |
| cNDA solution | 4 | Concentration of cNDA |
| | | solution: 50 mg/ml |
| DMSO | 2.5 | Final concentration in |
| | | reaction solution: 5% |
| Microorganism suspension | 5 | Concentration of |
| | | microorganism suspension: |
| | | 0.5 g (w.w)/ml |
| Total | 50 | |

TABLE 2
Conditions for HPLC analysis

| HPLC | LC 10-ADVP (Shimadzu) | | |
|---|---|---|---|
| Column | Xterra ™ RP18 (4.6 × 250 mm, Waters) | | |
| Detector | UV 240 nm | | |
| Column temp. | 40° C. | | |
| Flow rate | 1 ml/min. | | |
| Injection volume | 20 μl | | |
| | | 0.3% | |
| | Time (min.) | phosphoric acid | Acetonitrile |
| Mobile phase | 0 | 98 | 2 |
| | 5 | 92 | 8 |
| | 28 | 52 | 48 |
| | 30 | 20 | 80 |
| | 35 | 5 | 95 |
| | 36 | 98 | 2 |
| | 49 | 98 | 2 |

(2) Identification of Microorganism

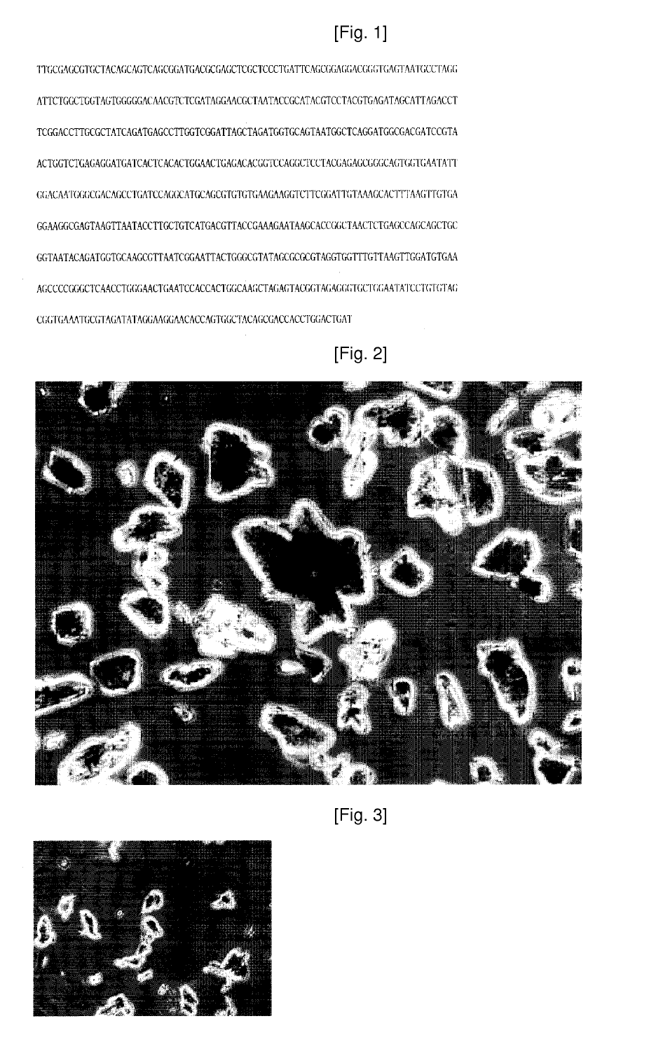

16S rDNA partial sequencing of the strain isolated in (1) was performed to identify the strain. The results are shown in FIG. 1 (SEQ ID NO: 1)

According to the results, the strain was identified as a bacterium belonging to the genus Pseudomonas. The strain was termed Pseudomonas sp. HN-72 and deposited at GenBank of the Korea Research Institute of Bioscience and Biotechnology (KRIBB), which is an international depository authority, under the accession number of KCTC-10819BP on Jun. 21, 2005. The morphology and biochemical characteristics of the strain (Pseudomonas sp. HN-72) were determined, and the results are summarized in Tables 3 and 4.

TABLE 3
Characteristics of Pseudomonas sp. HN-72

| Test | Characterization |
|---|---|
| Gram stain | Negative |
| Cell morphology | Rod |
| Optimal growth temperature | 25-30° C. |
| Oxidase | Positive |
| Denitrification | Negative |
| Gelatin degradation | Negative |
| Starch degradation | Negative |
| Catechol degradation | Negative |

TABLE 4
Utilization of carbon sources by Pseudomonas sp. HN-72

| | Utilization | | Utilization |
|---|---|---|---|
| Carbohydrate | ability | Carbohydrate | ability |
| Glucose | + | Lactase | + |
| Fructose | + | Citrate | + |
| Galactose | − | Glycerol | + |
| Arabinose | − | Phthalate | − |
| Rhamnose | + | Isopropanol | − |
| Trehalose | − | Butanol | + |
| Maltose | − | Sorbitol | − |
| Lactose | − | Mannitol | − |
| Sucrose | − | Ribose | + |
| Starch | − | Succinate | + |

Second Step: Purification of cNDA Using Microorganism

The Pseudomonas sp. strain HN-72 screened in the first step was inoculated onto 300 ml of an LB liquid medium and cultured in an incubator with shaking at 200 rpm and 30° C. for 16 hours. The culture was centrifuged to collect the bacteria. The collected bacteria were washed with a 0.85% physiological saline solution and suspended in 5 ml of a 0.85% physiological saline solution to activate the bacteria.

50 L of 50 mM potassium phosphate buffer was fed into a reactor A where a cNDA is purified by the microorganism. 1-10 kg of a cNDA was added to the reactor A and then KOH or NaOH was added thereto with stirring to adjust the pH of the reaction solution to 8.0. Water was added to the mixed solution to prepare a reaction solution for subsequent purification (cNDA concentration: 1-10%, FNA content in the cNDA: 0.01-4%).

0.2 to 10 kg/L of the active bacteria was fed into the reaction solution to react them with the reaction solution at 40° C. for 10 minutes to 2 hours while maintaining the temperature of the reaction solution at 40° C. As a result of the reaction, FNA contained in the cNDA was converted to NDA in the reactor A.

### Third Step: Crystallization of Purified cNDA

100 L of the solution of the purified cNDA, which was prepared in the second step, was transferred to a crystallization reactor C equipped with an agitator. After a sulfuric acid solution was added to the crystallization reactor to adjust the pH of the solution of the purified cNDA to 3.0, a crystallization reaction was conducted with stirring at a rate of 50 rpm for 30 minutes while maintaining the temperature at 40° C.

After completion of the crystallization, analysis of the crystallized cNDA was conducted using a microscope and a particle size analyzer. The analytical results reveal that the cNDA was satisfactorily crystallized without any aggregation between the individual crystal particles. The crystallized cNDA was measured to have an average particle size of about 160.7 □. FIG. 2 shows a micrograph of the crystallized cNDA.

Fourth Step: Washing and Drying of Crystallized cNDA

The slurry of the crystallized cNDA, which was prepared in the third step, was heated to above 220° C. using a preheater D, fed into a filtering/cleaning unit F, stirred at a pressure of 24.7 kg/cm² and a temperature of 225° C. for 30 minutes, and filtered. The filtrate (i.e. water) was discharged into a high-pressure filtrate collection unit G. 100 L of water at 225° C. from a solvent heating/supply unit E was added to the filtering/cleaning unit F, stirred under the same conditions as above for 30 minutes, and filtered. The filtrate (i.e. water) was discharged into the high-pressure filtrate collection unit G. This procedure was repeated a total of two times. 100 L of water at 225° C. from the solvent heating/supply unit E was added to the filtering/cleaning unit F and stirred for 30 minutes to homogeneously disperse pure NDA. The slurry containing the pure NDA was transferred to a high-pressure slurry collection unit H. After the pressure of the high-pressure slurry collection unit H dropped to ambient pressure, the slurry was transferred to a powder separation unit I (i.e. a decanter) where the water was removed from the slurry, followed by drying in a drying unit J at 120° C. to collect the NDA in a pure crystalline form.

### Example 2

NDA in a pure crystalline form was collected in the same manner as in Example 1, except that the microorganism was previously removed from the solution of the purified cNDA, which was prepared in the second step, using a polypropylene filter (pore size: 0.2 0) after the second step and prior to the third step.

### Examples 3 and 4

Pure NDA crystals were collected in the same manner as in Example 1, except that Bacillus sp. F-1 (KCTC-10342BP) and Bacillus sp. F-3 (KCTC-10335BP) were used instead of Pseudomonas sp. HN-72 (KCTC-10819BP), respectively.

### Examples 5 and 6

Pure NDA crystals were collected in the same manner as in Example 2, except that Bacillus sp. F-1 (KCTC-10342BP) and Bacillus sp. F-3 (KCTC-10335BP) were used instead of Pseudomonas sp. HN-72 (KCTC-10819BP), respectively.

The unpurified cNDA, the solution of the purified cNDA obtained after the second step (purification step), and the NDA in a pure crystalline form obtained after the fourth step (washing/drying step) in Example 1 were analyzed for the contents of the components therein. The analytical results are shown in Table 5.

TABLE 5
Component Analysis

| | | | After washing |
|---|---|---|---|
| | cNDA | After purification | and drying |
| NA | 0.046% | 0.040% | — |
| MNA | 0.085% | 0.082% | 0.002% |
| FNA | 0.210% | — | — |
| TMLA | 0.038% | 0.037% | 0.002% |
| Others | 0.130% | 0.102% | 0.006% |
| NDA | 99.491% | 99.739% | 99.990% |

From the results of Table 5, it could be confirmed that the purification method using the microorganism enabled substantially complete removal of impurities contained in the cNDA. Particularly, FNA was converted to NDA in a yield of 100%. As a result, high-purity 2,6-naphthalene dicarboxylic acid was produced with a purity of 99.9% or higher.

To determine optimal reaction conditions for the purification of the cNDA using the microorganism, particularly those for the crystallization of the solution of the purified cNDA, a series of experiments was conducted by varying reaction conditions as indicated in Tables 6 to 8, and the average particle sizes and recovery rates of cNDA crystals obtained after respective crystallization reactions according to the different reaction conditions were compared and analyzed.

### Experimental Example 1

### Crystallization Reactions with Varying Kinds of Acids and Stirring Rates

100 ml of the solution of the purified cNDA, which was prepared in the second step of Example 1, was added to reactors, each of which was equipped with an agitator, and then sulfuric acid, hydrochloric acid, glacial acetic acid and nitric acid solutions were added to the reactors to adjust the pH of the solutions of the purified cNDA to 3.0. The mixed solutions were stirred at different rates of 0, 50, 100, 200, 400, 800 and 1000 rpm. At this time, the temperature of the mixed solutions was maintained at 25° C. Under these reaction conditions, crystallization reactions were conducted for 30 minutes to obtain cNDA crystals.

After completion of the crystallization, analysis of the cNDA crystals was conducted using a microscope and a particle size analyzer. The analytical results reveal that the cNDA was satisfactorily crystallized without any aggregation between the individual crystal particles. FIGS. 3a, 3b and 3c show micrographs of the cNDA crystals obtained by adding the sulfuric acid solution to the reaction solutions of the purified cNDA and stirring the mixed solutions at different rates of 50 rpm, 200 rpm and 800 rpm, respectively. The average particle sizes of the cNDA crystals obtained under the respective conditions were measured, and the results are shown in Table 6. As can be seen from the data shown in Table 6, the cNDA crystals obtained after the addition of the sulfuric acid or hydrochloric acid solution and stirring at a rate of 0 to 400 rpm showed better results.

TABLE 6

| Acid | Sulfuric acid | | | | | | |
|---|---|---|---|---|---|---|---|
| Stirring rate (rpm) | 0 | 50 | 100 | 200 | 400 | 800 | 1,000 |
| Average particle | 117.2 | 121.3 | 116.8 | 112.0 | 100.6 | 40.7 | 22.6 |
| size (μm) | | | | | | | |
| Acid | Hydrochloric acid | | | | | | |
| Stirring rate (rpm) | 0 | 50 | 100 | 200 | 400 | 800 | 1,000 |
| Average particle | 118.0 | 120.7 | 117.2 | 110.4 | 98.5 | 38.7 | 19.0 |
| size (μm) | | | | | | | |
| Acid | Glacial acetic acid | | | | | | |
| Stirring rate (rpm) | 0 | 50 | 100 | 200 | 400 | 800 | 1,000 |
| Average particle | 72.6 | 87.0 | 74.3 | 69.2 | 48.6 | 12.0 | 5.8 |
| size (μm) | | | | | | | |
| Acid | Nitric acid | | | | | | |
| Stirring rate (rpm) | 0 | 50 | 100 | 200 | 400 | 800 | 1,000 |
| Average particle | 89.7 | 102.3 | 92.1 | 87.5 | 72.8 | 22.9 | 15.2 |
| size (μm) | | | | | | | |

### Experimental Example 2

### Crystallization Reactions with Varying Kinds of Acids and Temperatures

The procedure of Experimental Example 1 was repeated, except that the mixed solutions were stirred at a rate of 50 rpm at different reaction temperatures of 4, 15, 25, 40, 60 and 80° C.

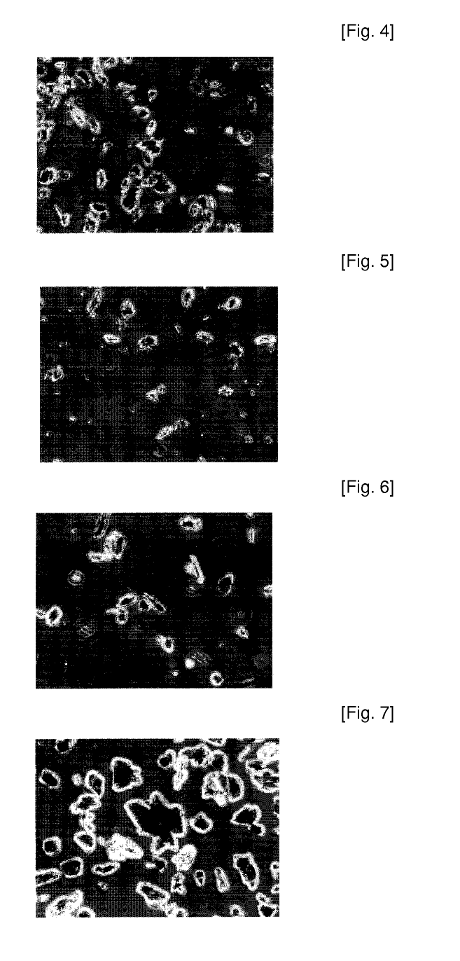

After completion of the crystallization, analysis of the cNDA crystals was conducted using a microscope and a particle size analyzer. The analytical results reveal that the cNDA was satisfactorily crystallized without any aggregation between the individual crystal particles. FIGS. 4a, 4b and 4c show micrographs of the cNDA crystals obtained by adding the sulfuric acid solution to the reaction solutions of the purified cNDA and stirring the mixed solutions at different reaction temperatures of 15° C., 40° C. and 80° C., respectively. The average particle sizes of the cNDA crystals obtained under the respective conditions were measured, and the results are shown in Table 7. As can be seen from the data shown in Table 7, the cNDA crystals obtained after the addition of the sulfuric acid or hydrochloric acid solution and stirring at a reaction temperature of 40° C. showed better results.

TABLE 7

| Acid | Sulfuric acid | | | | | |
|---|---|---|---|---|---|---|
| Temp. (° C.) | 4 | 15 | 25 | 40 | 60 | 80 |
| Average particle size (μm) | 108.2 | 116.2 | 120.8 | 160.7 | 149.3 | 136.8 |
| Acid | Hydrochloric acid | | | | | |
| Temp. (° C.) | 4 | 15 | 25 | 40 | 60 | 80 |
| Average particle size (μm) | 110.3 | 112.0 | 119.1 | 158.1 | 150.3 | 134.9 |
| Acid | Glacial acetic acid | | | | | |
| Temp. (° C.) | 4 | 15 | 25 | 40 | 60 | 80 |
| Average particle size (μm) | 46.3 | 60.7 | 76.3 | 82.6 | 60.3 | 48.1 |
| Acid | Nitric acid | | | | | |
| Temp. (° C.) | 4 | 15 | 25 | 40 | 60 | 80 |
| Average particle size (μm) | 64.9 | 88.3 | 108.6 | 132.9 | 112.9 | 95.4 |

### Experimental Example 3

### Recovery Rates at Different pH Values

The procedure of Experimental Example 1 was repeated, except that the mixed solutions were stirred at a rate of 50 rpm, a reaction temperature of 40° C. and different pH values of 1, 2, 3, 4, 5 and 6. After the crystallization reactions were conducted for 30 minutes, equal amounts of the reaction solutions were taken out of the respective reactors. The recovery rates of the cNDA crystals were measured, and the results are shown in Table 8. The results of Table 8 show that the recovery rates of the cNDA crystals were higher than 99% at a pH not higher than 3. The recovery rates of the cN DA crystals showed a tendency to increase with decreasing pH.

TABLE 8

| Acid | Sulfuric acid | | | | | |
|---|---|---|---|---|---|---|
| pH | 1 | 2 | 3 | 4 | 5 | 6 |
| Recovery rate (%) | 99.9 | 99.9 | 99.7 | 93.8 | 75.6 | 52.6 |
| Acid | Hydrochloric acid | | | | | |
| pH | 1 | 2 | 3 | 4 | 5 | 6 |
| Recovery rate (%) | 99.9 | 99.9 | 99.8 | 93.2 | 72.4 | 50.1 |
| Acid | Glacial acetic acid | | | | | |
| pH | 1 | 2 | 3 | 4 | 5 | 6 |
| Recovery rate (%) | 99.9 | 99.9 | 99.7 | 92.0 | 70.9 | 49.8 |
| Acid | Nitric acid | | | | | |
| pH | 1 | 2 | 3 | 4 | 5 | 6 |
| Recovery rate (%) | 99.9 | 99.9 | 99.7 | 93.6 | 67.5 | 42.6 |

### INDUSTRIAL APPLICABILITY

since a crude naphthalene dicarboxylic acid is purified and crystallized under respective suitable conditions using a microorganism capable of converting FNA to NDA, high-purity crystalline 2,6-naphthalene dicarboxylic acid can be advantageously produced on an industrial scale in an environmentally friendly manner.

1. A method for purifying a crude naphthalene dicarboxylic acid using a microorganism, the method comprising the steps of:
(a) reacting a microorganism having the ability to convert 2-formyl-6-naphthoic acid to 2,6-naphthalene dicarboxylic acid with a crude naphthalene dicarboxylic acid to remove 2-formyl-6-naphthoic acid contained in the crude naphthalene dicarboxylic acid;
(b) adding an acidic solution to the reaction solution prepared in step (a) to adjust the pH of the reaction solution and reacting the mixed solution with stirring to crystallize the crude naphthalene dicarboxylic acid;
(c) washing the crystallized crude naphthalene dicarboxylic acid to remove impurities contained in the crystallized crude naphthalene dicarboxylic acid; and
(d) drying the washed product to obtain 2,6-naphthalene dicarboxylic acid in a pure crystalline form.

2. The method according to claim 1, wherein step (a) includes the sub-steps of
1) inoculating the microorganism onto a liquid medium, culturing the inoculum with shaking, collecting the cultured bacteria by centrifugation, and suspending the collected bacteria in physiological saline or distilled water to activate the bacteria;
2) mixing a crude naphthalene dicarboxylic acid (cNDA) as a matrix with a buffer solution and adjusting the pH of the mixed solution by the addition of an alkaline solution to prepare a reaction solution for subsequent purification; and
3) reacting the active bacteria prepared in 1) with the reaction solution prepared in 2) to convert 2-formyl-6-naphthoic acid contained in the crude naphthalene dicarboxylic acid to 2,6-naphthalene dicarboxylic acid, so that the purity of the 2,6-naphthalene dicarboxylic acid is increased.

3. The method according to claim 2, wherein the microorganism is a bacterium belonging to the genus Bacillus or Pseudomonas.

4. The method according to claim 3, wherein the microorganism is Bacillus sp. F-I (KCTC-10342BP), Bacillus sp. F-3 (KCTC-10335BP), or Pseudomonas sp. HN-72 (KCTC-10819BP).

5. The method according to claim 2, wherein the buffer solution is selected from the group consisting of water, sodium carbonate buffer (Na₂O₃/NaHCO₃), glycine buffer (glycine/NaOH), potassium phosphate buffer (KH₂PO₄/KOH), sodium phosphate buffer (Na₂HPO₄/NaH₂PO₄), succinic acid buffer (succinic acid/NaOH), sodium acetate buffer (sodium acetate/acetic acid), citric acid buffer (citric acid/sodium citrate), sodium pyrophosphate buffer (Na₄P₂O₇/HCl), boric acid buffer (boric acid/NaOH), sodium borate buffer (sodium borate/HCl), and mixtures thereof; and has a concentration of 0.01 to 100 mM.

6. The method according to claim 2, wherein the alkaline solution is a NaOH or KOH solution.

7. The method according to claim 2, wherein the mixed solution further contains an organic solvent.

8. The method according to claim 7, wherein the organic solvent is selected from the group consisting of dimethylsulfoxide, N,N-dimethylformamide, N,N-dimethylacetamide, tetrahydrofuran, and mixtures thereof; and is added at a concentration of 0.01 to 10%.

9. The method according to claim 2, wherein the reaction is conducted at 25 to 50° C. for 1 minute to 2 hours.

10. The method according to claim 1, wherein the acidic solution is selected from the group consisting of sulfuric acid, hydrochloric acid, glacial acetic acid, nitric acid, and mixtures thereof.

11. The method according to claim 1, wherein the pH of the reaction solution is adjusted to the range of 1 to 4.

12. The method according to claim 1, wherein the reaction is carried out at 4° C. to 8O° C. for 1 minute to 12 hours.

13. The method according to claim 1, wherein the stirring is performed at a rate of 0 to 1,000 rpm.

14. The method according to claim 1, wherein step (c) is carried out by dispersing the crystallized cNDA in water, stirring the dispersion at a pressure of 1 to 28 kg/cm² and a temperature of 100 to 25O° C. for 10 minutes to 1 hour, filtering the crystallized cNDA to remove the water, and repeating the above procedure.

15. The method according to claim 1, wherein the drying is performed at 30 to 200° C.

16. The method according to claim 1, further comprising the step of removing the microorganism used in step (a) after step (a) and prior to step (b).

17. The method according to claim 16, wherein the removal of the microorganism is achieved by a microfilter system, a continuous type centrifugal separator or a decanter.

18. The method according to claim 17, wherein the microfilter system uses a filter having a pore size of 0.1 to 0.5 μm and made of a material selected from ceramics, stainless steel, polypropylene and polyethylene terephthalate (PET).

19. 2,6-Naphthalene dicarboxylic acid in a pure crystalline form produced by the method according to claim 1.

20. 2,6-Naphthalene dicarboxylic acid according to claim 19, wherein the 2,6-naphthalene dicarboxylic acid is in a regular or random crystalline form.

21. 2,6-Naphthalene dicarboxylic acid according to claim 19, wherein the regular crystalline form is a lattice structure.

22. 2,6-Naphthalene dicarboxylic acid according to claim 19, wherein the 2,6-naphthalene dicarboxylic acid crystal has an average particle diameter not smaller than 100 μm.
